(19) United States
(12) Patent Application Publication
(10) Pub. No.: US 2005/0153928 A1
(43) Pub. Date: Jul. 14, 2005

(54) Conjugates comprising a central nervous system-active drug linked to glucuronic acid or glucosamine through an amide bond and uses thereof

(76) Inventors: Michael F Holick, Sudbury, MA (US); Halasya Ramanathan, Mysore (IN)

Correspondence Address: STERNE, KESSLER, GOLDSTEIN & FOX PLLC, 1100 NEW YORK AVENUE, N.W., WASHINGTON, DC, 20005, US

(21) Appl. No.: 10/512,848

(22) PCT Filed: May 7, 2003
(86) PCT No.: PCT/US03/14050

Related U.S. Application Data
(60) Provisional application No. 60/378,333, filed on May 7, 2002.

(51) Int. Cl. A61K 31/728; A61K 31/7008

(52) U.S. Cl. 514/54; 514/62; 536/53; 536/55.2

(57) ABSTRACT

Disclosed are conjugates comprising a central nervous system-active drug linked through an amide bond to a glucuronic acid or glucosamine moiety, and uses thereof, e.g., for treating or ameliorating central nervous system diseases or disorders.

### BACKGROUND OF THE INVENTION

1. Field of the Invention

The present invention relates in general to the fields of medicine, pharmacology and biochemistry. More particularly, the invention relates to prodrugs capable of delivering a drug across the blood brain barrier and uses thereof.

2. Related Art

The delivery a drug to the central nervous system (CNS), e.g., the brain, is a challenging problem in the treatment of CNS disorders. The drug has to be transported across the selective filtering mechanism of the blood brain barrier (BBB) between the plasma and the CNS. In the past, the polar functional groups of drugs to be delivered to the brain have been masked as fat conjugates making the pro-drug more lipophilic. Gamma aminobutyric acid (GABA) that does not cross the blood brain barrier has been rendered active as a CNS drug by lipophilic conjugation (gabapentin; U.S. Pat. No. 4,894,476) and a similar fatty acid conjugation of dopamine is also known (U.S. Pat. Nos. 4,939,174; 6,107,499). Glycinated pro-drugs (e.g., valproic acid-glycine) are known to cross the blood brain barrier utilizing glycine transporters across the blood brain barriers. Rowley et al., J. Med. Chem., 40: 4053, 1997. Although it is well known that glucose transporters are present in the BBB, utilization of these transporters to get a drug across the BBB has not been relied upon to great extent.

Topiramate is an anti-convulsant comprising a sugar sulfarnate which is a fructo-pyranose derivative. Maryanoff et al., J. Med. Chem., 30: 88, 1987; Maryanoffet al., J. Med. Chem., 41: 1315, 1998.

Dopamine conjugated with glucose through a tethering agent has been shown to cross the BBB. Fernandez et al., Carbohydr. Res., 327: 353, 2000.

U.S. Pat. No. 5,977,326 discloses morphine-6-glucuronide compounds and processes for making the same.

U.S. Pat. Nos. 5,827,819 and 6,024,977 disclose biologically active compounds linked to polar lipid carrier molecules to enhance delivery across physiological barriers such as the BBB.

U.S. Pat. No. 6,313,106 discloses phospholipid derivatives of valproic acid for treating epilepsy, migraine, bipolar disorders and pain.

U.S. Pat. No. 5,051,448 discloses ester derivatives of GABA which cross the BBB.

U.S. Pat. No. 5,994,392 discloses anti-psychotic drugs conjugated to fatty acid carriers.

U.S. Pat. Nos. 4,595,695 and 5,162,573 disclose ester derivatives of valproic acid.

Many drugs have been conjugated to sugars for uses other than the treatment of CNS disorders.

U.S. Pat. No. 5,633,357 discloses methods for the synthesis of carboxylic acid glucuronides, e.g., tetrahydrocannabinoid carboxylic acid glucuronides.

U.S. Pat. No. 5,808,111 discloses 1-(D-glucopyranosyl)acitretinamide and 1-(D-glucopyranuronosyl)acitretinamide for cancer treatment.

U.S. Pat. No. 5,599,953 discloses C-glycoside analogs of N-(4-hydroxyphenyl) retinamid-O-glucuronide and N-glycoside analogs of retinoyl β-glucuronide for breast cancer treatment and prevention.

U.S. Pat. No. 6,339,060 discloses the specific targeting of biologically active compounds to specific sites by linking the compound to a microparticle with a linker that is non-specifically or specifically cleaved inside a mammalian phagocytic cell.

U.S. Pat. No. 5,760,072 discloses a paclitaxel prodrug coupled to a cleavable N-(aliphatic or aromatic)-O-glycosyl carbamate spacer group, wherein the prodrug is activated by a hydrolyzing enzyme, an endogenous enzyme or an exogenous enzyme.

U.S. Pat. No. 5,677,286 discloses glycosylated analogs of camptothecin for use as chemotherapeutic agents.

U.S. Pat. No. 4,855,463 discloses water soluble glucuronic acid derivatives of Vitamin A.

U.S. Pat. No. 5,340,803 discloses conjugates of a cytotoxic compound which is a substrate for tyrosinase and glucuronic acid for the treatment of tumor cells which have β-glucuronidase and tyrosinase activity.

U.S. Pat. No. 5,561,119 discloses glycosylated prodrugs and their use with tumor-specific immunoenzymatic conjugates for the treatment of cancer.

U.S. Pat. No. 5,559,235 discloses water soluble derivatives of camptothecin for treatment of cancer.

U.S. Pat. No. 6,043,367 discloses cancer treating conjugates of a glucuronide and a cytotoxic agent joined by an electron-transporting linker.

U.S. Pat. No. 6,218,519 discloses conjugates of an anthracyclinone group with ester, gylcoside or glucuronide structures which are hydrolyzed by the corresponding esterase, glycosidase or glucuronidase for inhibition of tumor cells and bacterial growth.

U.S. Pat. No. 6,166,089 discloses prodrugs which are covalent conjugates of a pharmacologically active compound and an intracellular transporting adjuvant, characterized by the presence of a covalent bond which is scission-sensitive to intracellular enzyme activity, preferably lipase activity.

A large number of drugs and their metabolites are conjugated in the body as part of the elimination pathway. Glucuronic acid is the most frequent partner to the drug in conjugation. Remington's Pharmaceutical Sciences, A. Osol et al. (eds.), pp. 677 (1980).

According to the present invention, by linking a CNS-active drug to a sugar using an amide bond to form a prodrug, the prodrug will utilize glucose transporters present in the BBB to enter the CNS and be activated by endogenous amidases, thus enhancing treatment of CNS diseases and disorders.

### SUMMARY OF THE INVENTION

The invention relates to compounds that are conjugates of a CNS-active drug linked through an amide bond to a sugar moiety, with or without a linker moiety. In particular, drugs containing an amino group are linked to a carboxyl group on glucuronic acid and drugs containing a carboxyl group are linked to an amino group on glucosamine in order to form the amide bond. The conjugates act as prodrugs which are able to cross the BBB by utilizing glucose transporters and enter the CNS where the drugs are activated through enzymatic removal of the sugar moiety by endogenous amidases.

The invention also relates to a method for the treatment or amelioration of CNS diseases, disorders or conditions.

The invention also relates to pharmaceutical compositions comprising the compounds of the invention and a pharmaceutically acceptable carrier.

### DETAILED DESCRIPTION OF THE INVENTION

CNS-Active Drugs

CNS-active drugs are biologically active compounds which exert a useful effect on the CNS when administered to an animal. CNS-active drugs include compounds that are effective for the treatment, amelioration or prevention of CNS diseases, disorders or conditions. These diseases, disorders or conditions encompass neurological and psychiatric disorders, including but not limited to, Alzheimer's disease, Parkinson's disease, Huntington's disease, seizures/epilepsy, Tourette Syndrome, attention deficit hyperactivity disorder, headache, migraine, stroke, trigeminal neuralgia, depression, sleep disorders and trauma. Also included are compounds which effect the CNS in desirable ways that are not related to diseases or disorders, e.g., for appetite suppression. Examples of such CNS-active drugs include, but are not limited to, dopamine, valproic acid, GABA, tacrine, phenyloin, carbamazapine, phenobarbital, primidone, clonazapam, felbamate, topiramate, tiagibine, methylphenidate, amphetamine, dextroamphetamine, methamphetamine, pemoline, desipramine, nortriptyline, bupropion, clonidine, guanfacine, pimozide, sumatriptan, zolmitriptan, rizatriptan, baclofen, levodopa, carbidopa, ropinirole, bromocriptine, pergolide, pramipexole carbamazepine, lainotrigine, levetiracetam zonisamide, galantamine, serotonin, melatonin, sitalin, fluoxetine and amantadine.

Sugar Residues

Sugar residues that are useful in the practice of the present invention include glucosamine, glucuronic acid, hyalobiuronic acid and hyaluronic acid. Other sugar residues that may be used in the practice of the invention include derivatives of glucosamine and glucuronic acid and their mono fluoro derivatives. Preferably, endogenous anidases will recognize and cleave the sugar derivative-drug bond, thus releasing the drug. The sugar residues may have free hydroxy groups, or the hydroxy groups may be acylated, e.g. with a group R₁—(C═O)—, wherein R₁ is hydrogen, C₁₋₆ alkyl, C₆₋₁₀ substituted or unsubstituted aryl or C₇₋₁₆ aralkyl. Preferably, the acyl groups are acetyl or propionyl. Other preferred R₁ groups are phenyl, nitrophenyl, halophenyl, lower alkyl substituted phenyl, lower alkoxy substituted phenyl and the like or benzyl, lower alkoxy substituted benzyl and the like.

The sugar residues may be fully or partially acylated or completely deacylated. The completely or partially acylated glycoside is useful as a defined intermediate for the synthesis of the deacylated material. Useful protecting groups include, but are not limited to, acetyl, benzoyl, nicotinoyl, benzyl, methyl and phenyl.

The compounds of the invention may be in the form of an acid/amine addition salt by treatment with an inorganic or organic acid/base.

Methods of Making the Compounds of the Invention

CNS-active drugs may be linked to a sugar moiety either directly or with the use of a linker moiety. For direct linkage, drugs containing an amino group are linked to a carboxyl group on a sugar such as glucuronic acid and drugs containing a carboxyl group are linked to an amino group on a sugar such as glucosamine in order to form the amide bond. When a linker moiety is used, the CNS-active drug and the sugar moiety are linked through a linker moiety such that an amide bond is formed between the drug and the linker moiety and/or between the sugar and the linker moiety. In one embodiment of the invention, the linker moiety is an alkylene dicarboxylic acid, e.g., malonic acid, succinic acid, glutaric acid, adipic acid or the like. According to the present invention, when the CNS-active drug is dopamine, the dopamine is directly linked to the sugar moiety.

Glucuronate/drug conjugates may be prepared by condensing protected glucuronic acid together with a drug containing an amino group. Similarly, protected glucosamine may be conjugated with a drug containing a carboxyl group and deprotected.

In one particular embodiment, protected D-glucosylamine may be prepared for amide formation with carboxylic acid as shown in Scheme 1.

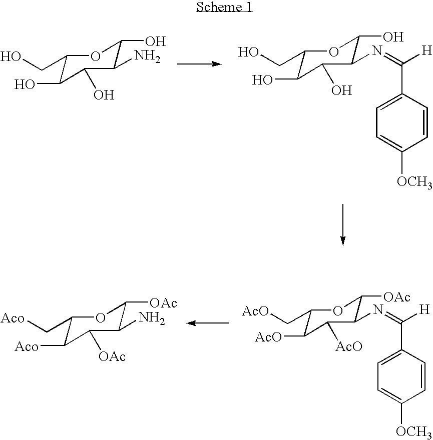

In another embodiment, protected D-glucuronoyl chloride may be prepared for amide formation with an amine as shown in Scheme 2.

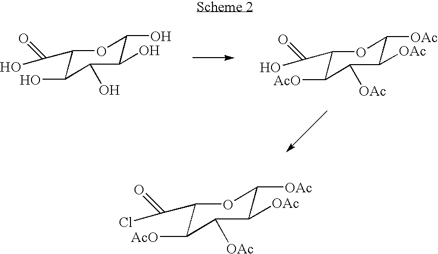

Similarly, N-pthalimidobutyroyl-D-glucosamide may be prepared as shown in Scheme 3.

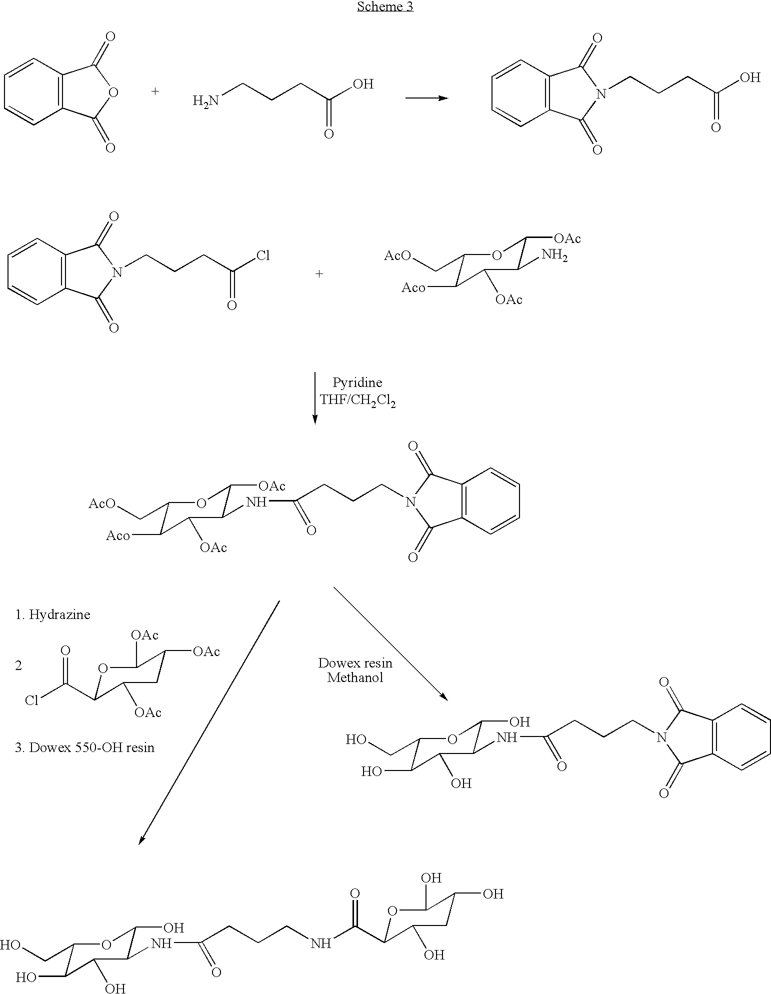

The preparation of various conjugates of the present invention using the protected sugars shown above are depicted below.

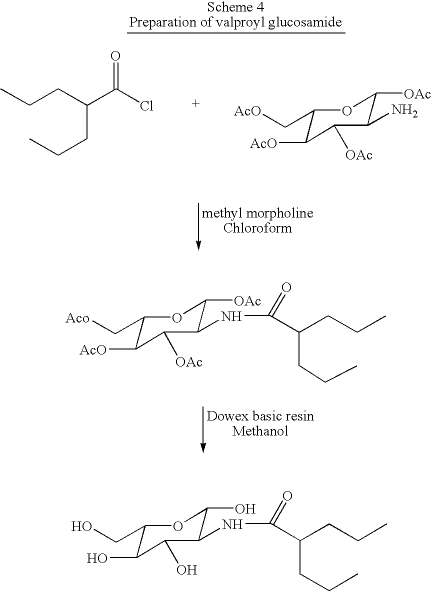

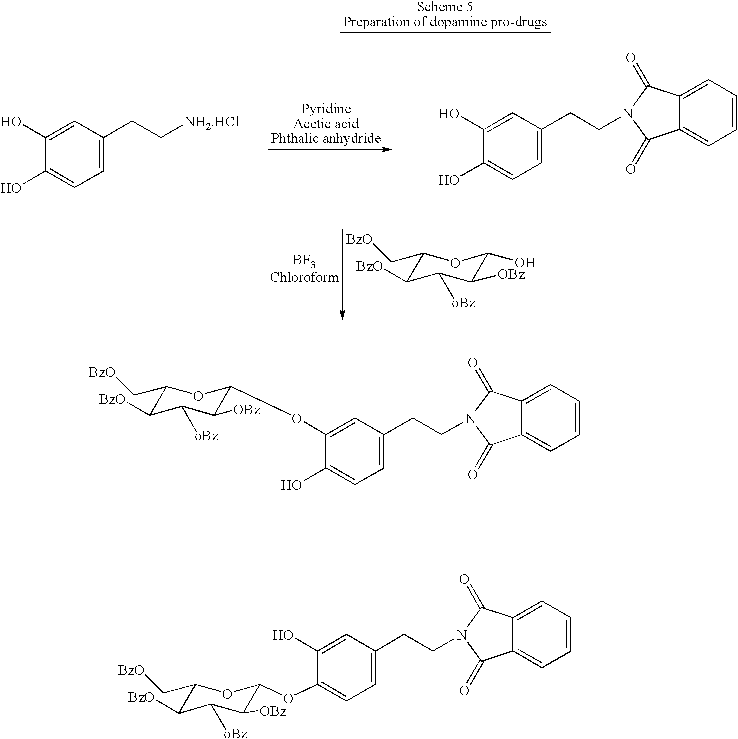

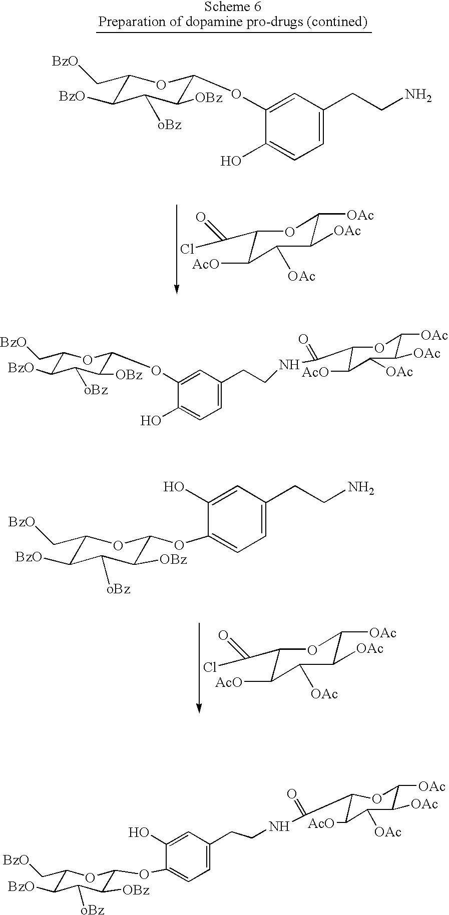

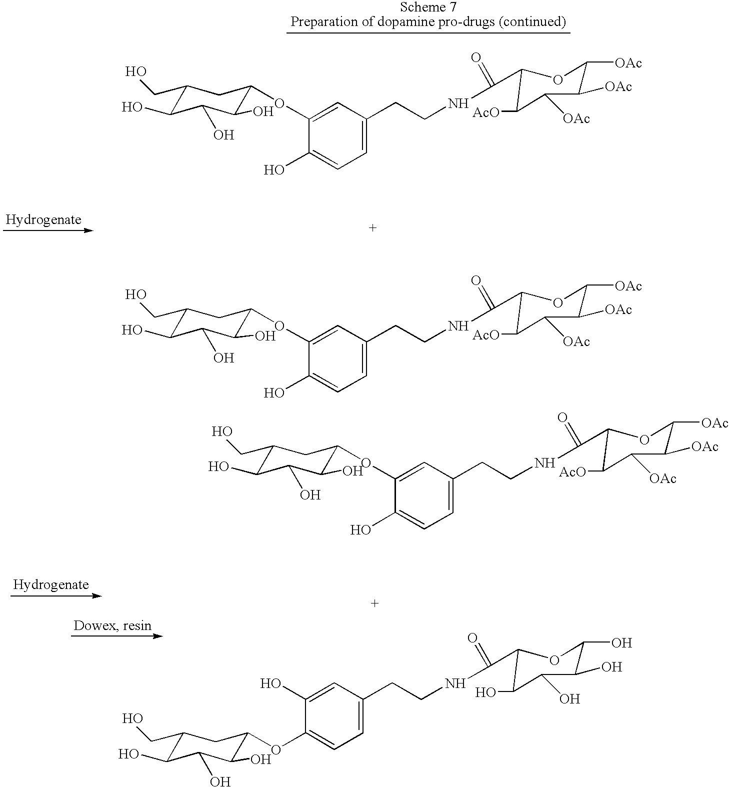

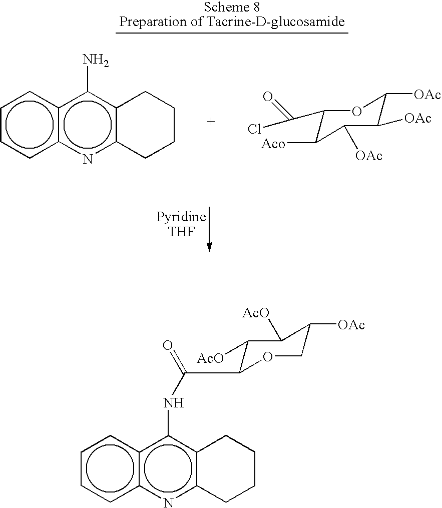

### EXPERIMENTAL

Synthesis of N-β-methoxybenzylidene)-D-glucosamine

D-glucosamine hydrochloride (215 g; 1 mol) was dissolved in sodium hydroxide solution (1 N; 1 liter) and p-anisaldehyde (122 ml) was added. The solid product obtained was filtered off and dried. The product (250 g) had a melting point of 165° C. in accordance with the literature.

Synthesis of N-(p-methoxy-benzylidene)-1,3,4,6-tetra-O-acetyl-D-glucosamine

The p-anisylidene derivative obtained above (250 g) was dissolved in pyridine (1.25 ml) and acetic anhydride (750 ml) was added slowly at room temperature. The mixture was stirred for 12 hours at room temperature and the clear solution was poured into crushed ice/water mixture (5 liters) and filtered. The precipitate was filtered off and crystallized from methanol (270 g). The product had a melting point of 180-1° C. in accordance with the literature.

Synthesis of 1,3,4,6-tetra-O-acetyl-D-glucosamine Hydrochloride

To a boiling solution of tetra-O-acetyl-p-anisylidene derivative (150 g) obtained as above in acetone (750 ml) was added hydrochloric acid (5 N, 62.5 ml). After stirring the mixture mechanically for 15 minutes, the product was isolated by cooling and adding ether (100 ml) to facilitate complete precipitation. The precipitate was filtered and washed once with ether and dried (100 g; m.p.=230° C. as reported).

Proton NMR spectrum in CDCl₃: δ 2.2-2.0 (overlapping singlets; 12-H; acetyl); δ 3.0 (t; 1-H; sugar-H); δ 3.8 (broad singlet; 1-H; sugar-H); δ 4.1 (d; 1-H; sugar-H); δ 4.3 (d; 1-H; sugar-H); δ 5.0 (m; 2-H; sugar-H) and δ 5.4 (doublet; 1-H; anomeric-1).

Preparation of 1,2,3,4-tetra-O-acetyl-D-glucuronic Acid:

Glucuronic acid (5 g) was added to a cooled solution of pyridine (15 ml). Acetic anhydride (25 ml) was added and the solution stirred at ambient temperature for 24 hours. The mixture was poured into ice cold water (500 ml). The gummy solid that separated out was decanted and extracted into chloroform (200 ml) and dried over magnesium sulfate. Evaporation of the solvent after charcoal treatment and re-crystallization of the residue gave white crystals from ethanol (8 g).

Proton NMR spectrum in CDCl₃: δ 6.2 (s; 1-H; C₅—H); δ 5.0-5.3 (m; 4-H; sugar-H); and δ 2.0-2.2 (overlapping singlets; 12-H; acetyl).

Preparation of 1,2,3,4-tetra-O-acetyl-D-glucuronoyl Chloride

1,2,3,4-tetra-O-acetyl glucuronic acid (8 g) was dissolved in chloroform (50 ml) and oxalyl chloride (10 ml; excess) was added slowly at 5° C. and allowed to stir and warm to room temperature. After the cessation of gas evolution, the solution was heated gently to reflux and cooled. Solvents and excess oxalyl chloride were removed under low pressure and the product used as is in reacting with tacrine as below.

Preparation of 1′,2′,3′,4′-tetra-O-acetyl-D-glucuronoyl-(1,2,3,4,-tetrahydro)-9-acrinamide (9-[1′,2′,3′,4′-tetra-O-acetyl-D-glucuronamido]-1,2,3,4-tetrahydro Acridine)

A solution of tacrine hydrochloride in pyridine (10 ml) and dimethylformamide (20 ml) at 5° C. was stirred at inert atmosphere. 1,2,3,4-tetra-O-acetyl-D-glucuronoyl chloride (8 g; excess) was added and stirred at room temperature for 12 hours. The mixture was poured into water (200 ml) containing saturated sodium bicarbonate (50 ml). The product was extracted into ethyl acetate (3 times 250 ml) and the combined organic portion was washed well to remove pyridine and dried over magnesium sulfate. The product was separated from unreacted starting materials by column chromatography on silica gel using ethyl acetate-methanol mixtures. The purification afforded white crystals of titled compound (3.2 g; re-crystallized from acetone).

Proton NMR spectrum in CD₃OD: δ 7.5-8.1 (multiplets; 4-H; aromatic-H); δ 5.9 (broad singlet; C₅—H); δ 3.6-5.3 (multiplets; sugar-H; 4-H); and δ 1.65-2.5 (multiplets; CH₂; 8-H and 12-H from acetate).

Preparation of 4-Phthalimidobutanoic Acid

4-Aminobutyric acid (GABA; 103 g; 1 mol) was charged into a 2 liter reactor equipped with a mechanical stirrer and phthalic anhydride (148 g; 1 mol) and glacial acetic acid (250 ml) were charged. The contents were heated to reflux and stirred for 2.5 hours and cooled to room temperature and stirred. Water (1.5 L) was added and the mixture stirred and cooled to 5° C. After 1 hour at 5° C., the slurry was filtered and the cake was washed with water (500 ml). The product was air dried and it afforded 175 g of white powder.

Preparation of 3-Phthalimidopropanoic Acid

In a similar fashion 3-aminopropanoic acid was converted to the titled compound as a white crystalline powder in 78% yield.

Proton NMR spectrum in CDCl₃: δ 1.3 (broad singlet; amine-H; 2-H); a (2.7; triplet; CH₂; 2H); δ (3.9; triplet; N—CH₂; 2H) and δ (7.7-7.9; doublet; aromatic-H; 4H).

Preparation of N-phthalimidobutanoyl Chloride

In a well ventilated fume-hood N-phthalimidobutyric acid (23.3 g) was added to a solution of toluene (100 ml) containing thionyl chloride (9.0 ml) and dimethylformamide (0.5 ml). The mixture was heated to 45-50° C. and maintained till the gas evolution ceased. The mixture was stirred and heated for a period of 1 hour more. Toluene was removed in a rotary evaporator below 50° C. The resulting paste was redissolved in dichloromethane (50 ml) and evaporated to remove trace amounts of thionyl chloride. The resulting product was connected to a high vacuum pump and used as such in the next step. It was a low melting solid.

Preparation of N-Phthalimidobutanoyl-1,3,4,6-tetra-O-acetyl-2-glucosamide

In a 50 ml round bottomed flask, thionyl chloride (0.93 ml; 8.57 mmol) were added to a solution of 4-N-phthalimidobutyric acid (2 g; 8.58 mmol) in 40 ml toluene and a few drops of DMF. The reaction mixture was allowed to stir for 3.5 hours at room temperature. The product was isolated as a solid after removing toluene at 45-50° C. by rotary evaporation and toluene (20 ml) was added again and evaporated to remove trace amounts of thionyl chloride.

To a stirred solution of 1,3,4,6-tetra-O-acetylglucose (4.47 g; 12.9 mmol) in tetrahydrofuran (60 ml) were added to a solution of N-phthalimidobutanoyl chloride (2.25 g; 8.58 mmol) in tetrahydrofuran (20 ml) followed by pyridine (2 ml). The resulting solution was stirred at room temperature for 4 hours. The product was quenched with saturated sodium bicarbonate solution (150 ml). The desired compound was extracted with dichloromethane (3 times 100 ml) and dried over magnesium sulfate. The crude product was isolated after evaporation and thin layer chromatography of the product (using 1:19 methanol:dichloromethane mixture) showed no signs of starting material. The desired glucosamide was purified by acetone crystallization to afford 4.48 g of white crystals in 92.8% yield.

Proton NMR spectrum in CDCl₃ (CD₃OD mixture): δ 7.7-7.8 (two singlets; Ar—H; 4H); δ 6.1 (d; 1H; sugar-H); δ 5.8 (d; 1-H; sugar-H); δ 5.1-5.3 (two overlapping triplets; 2-H; sugar-H) δ 3.6-4.3 (multiplets; 6-H; sugar-H and N—CH₂); and δ 1.9-2.2 (multiplet and overlapping singlets; 16-H; acetyl and 2×CH₂).

Preparation of 2-(n-propyl)-pentanoyl-1′,3′,4′,6′-tetra-O-acetyl-2′-glucosamide (valproyl-2-glucosamide)

To a solution of valproic acid (2.21 ml; 15.35 mmol) in chloroform (10 m) was added thionyl chloride (2.2 ml; 15.3 mmol). The reaction mixture was allowed to stir for 2 hours at room temperature until the gas evolution ceased. The acid chloride was isolated by distilling off chloroform and excess thionyl chloride. Chloroform (20 ml) was added and distilled to remove further traces of thionyl chloride. Dichloromethane (10 ml) was added to the residual acid chloride and used as such as below.

To a cooled solution of N-methyl morpholine (6 ml) and tetrahydrofuran (60 ml) was added 1,3,4,6,-tetra-O-acetyl-D-glucosamine (3.96 g; 11.5 mmol). To this stirred mixture was added the acid chloride in dichloromethane from above. The mixture was stirred for 14 hours at room temperature. The product was isolated after pouring into saturated sodium bicarbonate (150 ml) and extracting with dichloromethane (3 times 100 ml). The organic portion was dried over magnesium sulfate and evaporated. A solid product was obtained. Thin layer chromatographic examination showed the absence of tetra-O-acetyl glucosamine. The solid product was re-dissolved in chloroform and crystallized from ether to a white solid (3 g; 60% isolated yield).

Proton NMR spectrum in CDCl₃: δ 0.8-1.5 (multiplet; 14H; aliphatic-H); δ 2.0-2.2 (overlapping singlets; 12-H; acetyl); δ 3.0 (s; 1-H; sugar-H); δ 3.7 (broad singlet; 1-H; sugar-H); δ 4.1 (d; 1-H; sugar-H); δ 4.3 (m; 1-H; sugar-H); δ 5.0 (m; 2-H; sugar-H) and δ 5.4 (d; 1-H; anomeric hydrogen).

N-phthalimido-3-hydroxytyramine (N-phthalimido dopamine):

To a stirred suspension of acetic acid (25 ml) and dopamine hydrochloride (5 g) was added pyridine (20 ml) and phthalic anhydride (4.2 g). The mixture was stirred and heated to reflux for 1 hour and cooled, then poured into water (200 ml) and filtered. The precipitate was washed twice with saturated sodium bicarbonate solution (20 ml) and water (20 ml). The dried gelatinous yellow solid (3.5 g) was used as is for the next step.

Proton NMR spectrum in CDCl₃: δ 2.6 (broad singlet; 2-H; benzylic-H); δ 3.7 (broad singlet; 2-H; N—CH₂); δ 6.4-6.6 (two weakly split singlets; 3-H; aromatic-H from catechol); and δ 7.5-7.7 (two singlets; 4-H; aromatic-H from phthalimide).

Glucosylation of N-phthalimido-3-hydroxy tyramine (N-phthalmido Dopamine)

To a stirred suspension of N-phthalimido-3-hydroxy tyramine (3 g) in chloroform (35 ml) and acetonitrile (10 m) was added molecular sieves (4° A; 10 g) and stirred at 5° C. under argon. After 30 minutes, boron trifluoride-etherate (1.2 m) was added and the lightly colored solution was stirred for 5 minutes and tetra-O-benzyl glucopyranose (6 g) was added in chloroform (10 ml) in one lot. The mixture stirred at room temperature to a clear lightly purple solution during 1 hour. The mixture was stirred for a further period of 12 hours at room temperature and extracted with chloroform (250 ml), washed with saturated sodium bicarbonate (150 ml) followed by water (100 ml) and dried over magnesium sulfate. Upon evaporation and silica gel column chromatography eluting with dichloromethane and methanol mixtures, the products were separated. Two major products were obtained which are isomeric glucosides.

The isomeric mixtures were separated by column chromatography on silica gel using dichloromethane, methanol and ethylacetate mixtures. The proton NMR spectra of the isomeric mixtures were identical due to the complexity of the benzyl protecting groups in the sugar region.

Proton NMR spectrum in CDCl₃: δ 2.8 (multiplet, 2-H, dopamine-CH₂); δ 3.3-5.2 (complex; 17-H; benzylic-CH₂, sugar-H); δ 6.9-7.7 (complex; 23-H; Ar—H).

Methods of Use and Formulation

Particularly preferred routes of administration of the compounds of the present invention are per os, such as elixirs, tablets and capsules, as exemplified below, and by i.v. administration.

More generally, the compounds of the present invention can be administered in any appropriate pharmaceutically acceptable carrier for oral administration since the compounds are biologically active upon oral administration. The compounds of the invention may also be administered in any appropriate pharmaceutical carrier for parenteral, intramuscular, transdermal, intranasal, buccal or inhalation administration. They can be administered by any means that treat or ameliorate the conditions and diseases described herein.

The dosage administered will depend on the age, health and weight of the recipient, kind of concurrent treatment, if any, frequency of treatment and the nature of the effect desired. An exemplary systemic daily dosage is about 0.1 mg to about 500 mg. Normally, from about 1.0 mg to 100 mg daily of the compounds, in one or more dosages per day, is effective to obtain the desired results. One of ordinary skill in the art can determine the optimal dosages and concentrations of active compounds with only routine experimentation.

The compounds can be employed in dosage forms such as tablets and capsules for oral administration. Such dosage forms may comprise well known pharmaceutically acceptable carriers and excipients. In a preferred embodiment, the dosage forms comprise cyclodextran and/or other saccharides and/or sugar alcohols. The compounds may also be formulated in a sterile liquid for formulations such as solutions (e.g. in saline) or suspensions for parenteral use. A lipid vehicle can be used in parenteral administration.

The compounds could also be administered via topical patches, ointments, gels or other transdermal applications. In such compositions, the active ingredient will ordinarily be present in an amount of at least 0.001% by weight based on the total weight of the composition, and not more than 50% by weight. An inert pharmaceutically acceptable carrier is preferable such as 95% ethanol, vegetable oils, propylene glycols, saline buffers, sesame oil, etc. Remington's Pharmaceutical Sciences, 18^{th} Edition, Geiinaro et al. (eds.), 1990, exemplifies methods of preparing pharmaceutical compositions.

The compounds may also be employed in fast dissolving dosage forms, as described in U.S. Pat. No. 6,316,027, comprising the compounds of the invention, water, gelatin and other ingredients.

The compounds of the invention may be formulated as part of a liposomal composition.

Topical formulations for transdermal, intranasal or inhalation administration may be prepared according to methods well known in the art. For topical administration, the compounds may be applied in any of the conventional pharmaceutical forms. For example, the compounds may be administered as part of a cream, lotion, aerosol, ointment, powder, drops or transdermal patch. Ointments and creams may, for example, be formulated with an aqueous or oily base with the addition of suitable thickening and/or gelling agents. Such bases may include water and/or an oil such as liquid paraffin or a vegetable oil such as peanut oil or castor oil. Thickening agents which may be used include soft paraffin, aluminum stearate, cetostearyl alcohol, polyethylene glycols, wool-fat, hydrogenated lanolin, beeswax and the like.

Lotions may be formulated with an aqueous or oily base and will in general also include one or more of a stabilizing agent, thickening agent, dispersing agent, suspending agent, thickening agent, coloring agent, perfume and the like.

Powders may comprise any suitable powder base including talc, lactose, starch and the like. Drops may comprise an aqueous or non-aqueous base together with one or more dispersing agents, suspending agents, solubilizing agents and the like.

The compositions may further comprise one or more preservatives including bacteriostatic agents including methyl hydroxybenzoate, propyl hydroxybenzoate, chlorocresol, benzalkonium chloride and the like.

The topical compositions comprise from about 0.0001% to 5% by weight, preferably, 0.001 to 0.5% by weight, more preferably, 0.01 to 0.25% by weight of the active compounds.

The compounds of the invention are substantially pure. The phrase “substantially pure” encompasses compounds created by chemical synthesis and/or compounds substantially free of chemicals which may accompany the compounds in the natural state, as evidenced by thin layer chromatography (TLC) or high performance liquid chromatography (HPLC).

Animals which may be treated according to the methods of the present invention include all animals which may benefit therefrom. Included in such animals are humans, veterinary animals and pets, although the invention is not intended to be so limited.

From the foregoing description, one skilled in the art can easily ascertain the essential characteristics of this invention, and without departing from the spirit and scope thereof, can make various changes and modifications of the invention to adapt it to various usages and conditions without undue experimentation. All patents, patent applications and publications cited herein are incorporated by reference in their entirety.

1. A compound comprising a central nervous system (CNS)-active drug linked to a sugar moiety selected from the group consisting of glucuronic acid, glucosamine, hyalobiuronic acid and hyaluronic acid through an amide bond with or without a linker moiety, provided that when the CNS-active drug is dopamine, it is directly linked to the sugar moiety.

2. The compound of claim 1, wherein an amino group on the CNS-active drug and a carboxyl group on glucuronic acid are linked to form the amide bond.

3. The compound of claim 1, wherein a carboxyl group on the CNS-active drug and an amino group on glucosamine are linked to form the amide bond.

4. The compound of claim 1, wherein said glucosamine comprises an amino group at the 1-position or the 2-position.

5. The compound of claim 1, wherein said CNS-active agent is effective for the treatment or amelioration of Alzheimer's disease, Parkinson's disease, Huntington's disease, seizures/epilepsy, Tourette Syndrome, attention deficit hyperactivity disorder, headache, migraine, stroke, trigeminal neuralgia, depression, weight-loss, sleep-disorders or trauma.

6. The compound of claim 1, wherein said CNS-active agent is selected from the group consisting of dopamine, valproic acid, gamma-aminobutyric acid and tacrine.

7. The compound of claim 1, wherein said compound is valproyl-2-glucosamide.

8. The compound of claim 1, wherein said compound is glucosylated dopamine.

9. The compound of claim 1, wherein said compound is tacrine-D-glucosamide.

10. A pharmaceutical composition comprising the compound of claim 1 and a pharmaceutically acceptable carrier.

11. A method for the treatment or amelioration of a central nervous system disease, disorder or condition in an animal, comprising administering to an animal in need thereof an effective amount of the compound of claim 1, such that the disease, disorder or condition is treated or ameliorated.

12. The method of claim 11, wherein said compound is administered as part of a pharmaceutical composition comprising a pharmaceutically acceptable carrier therefor.

13. The method of claim 11, wherein said animal is a human.
